(19) United States
(12) Patent Application Publication
(10) Pub. No.: US 2020/0143920 A1
(43) Pub. Date: May 7, 2020

(54) SYSTEMS FOR FACILITATING THE MANAGEMENT OF HEALTHCARE DELIVERY PROCESSES

(71) Applicant: James Crosby, Scottsdale, AZ (US)

(72) Inventor: James Crosby, Scottsdale, AZ (US)

(21) Appl. No.: 16/677,475

(22) Filed: Nov. 7, 2019

Related U.S. Application Data
(60) Provisional application No. 62/757,028, filed on Nov. 7, 2018.

(51) Int. Cl. G16H 10/65; G16H 40/67

(52) U.S. Cl. CPC G16H 10/65; G16H 40/67

(57) ABSTRACT

Disclosed herein is a system for facilitating the management of healthcare delivery processes, in accordance with some embodiments. Accordingly, the system may include a communication device configured for receiving at least one personal medical data from at least one personal device. Further, the communication device may be configured for transmitting at least one notification to at least one of a healthcare provider device of at least one healthcare provider device and a personal device of the at least one personal device. Further, the system may include a processing device configured for analyzing the at least one personal medical data. Further, the processing device may be configured for managing at least one healthcare delivery process based on the analyzing. Further, the system may include a storage device configured for storing the at least one notification and the at least one personal medical data in a database.

The current application claims a priority to the U.S. provisional patent application Ser. No. 62/757,028 filed on Nov. 7, 2018.

### FIELD OF THE INVENTION

Generally, the present disclosure relates to the field of data processing. More specifically, the present disclosure relates to systems for facilitating the management of healthcare delivery processes.

### BACKGROUND OF THE INVENTION

Existing techniques for facilitating the management of healthcare delivery processes are deficient with regard to several aspects. For instance, current technologies do not provide adequate interaction between patients and healthcare providers. Further, current technologies do not provide patient accountability. Further, the current technologies do not the patient to promptly receive healthcare due to lengthy process involved in requesting healthcare. Moreover, current technologies do not provide medical history associated with the patient at a new medical facility. Furthermore, the current technologies do not monitor biometrics and vitals (such as blood pressure, blood oxygen levels, pulse, etc.) associated with the patient to identify a potential health threat. Further, the patients are often left in the dark regarding the obligations and/or benefits associated with healthcare, such as medical bills, due to the lack of a clear channel of communication between the healthcare providers and the patients. Further, there are few or limited resources that allow for the continuous monitoring of people's health, leaving the responsibilities to patients who oftentimes do not have the knowledge or experience to recognize health threats. In addition, people's medical history is often restricted and hard to access between different healthcare providers.

Therefore, there is a need for improved methods, systems, apparatuses and devices for facilitating the management of healthcare delivery processes that may overcome one or more of the above-mentioned problems and/or limitations.

### BRIEF SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a system for facilitating the management of healthcare delivery processes, in accordance with some embodiments. Accordingly, the system may include a communication device configured for receiving at least one personal medical data from at least one personal device. Further, the communication device may be configured for transmitting at least one notification to at least one of a healthcare provider device of at least one healthcare provider device and a personal device of the at least one personal device. Further, the system may include a processing device configured for analyzing the at least one personal medical data. Further, the processing device may be configured for managing at least one healthcare delivery process based on the analyzing. Further, the system may include a storage device configured for storing the at least one notification and the at least one personal medical data in a database.

Further, disclosed herein is a system for facilitating the management of healthcare delivery processes, in accordance with some embodiments. Accordingly, the system may include a communication device configured for receiving at least one personal medical data from at least one personal device. Further, the communication device may be configured for transmitting at least one notification to at least one of a healthcare provider device of at least one healthcare provider device and a personal device of the at least one personal device. Further, the system may include a processing device configured for analyzing the at least one personal medical data. Further, the processing device may be configured for managing at least one healthcare delivery process based on the analyzing. Further, the system may include a storage device configured for storing the at least one notification and the at least one personal medical data in a database.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating the management of healthcare delivery processes, in accordance with some embodiments

FIG. 3 is a block diagram of a system for facilitating the management of healthcare delivery processes, in accordance with some embodiments.

FIG. 4 is a flowchart of a method to facilitate authentication of a signal associated with a wearable or implantable device, in accordance with some embodiments.

FIG. 5 is a flowchart of a method to facilitate communication between the at least one patient and the authority, in accordance with some embodiments.

FIG. 6 is a flowchart of a method to facilitate communication between the at least one patient and the authority to obtain the details of the at least one patient to provide treatment, in accordance with some embodiments.

FIG. 7 is a flowchart of a method to facilitate registration of the at least one patient, in accordance with some embodiments.

FIG. 8 is a flowchart of a method to facilitate the retrieving of the details of the at least one patient from the wearable or implantable device, in accordance with some embodiments.

FIG. 9 is a flowchart of a method to facilitate compiling and recording of treatment processes associated with health care of the at least one patient, in accordance with some embodiments.

FIG. 10 is a block diagram of a system for facilitating the exchange of data over a plurality of data transfer protocols and standards, in accordance with some embodiments.

FIG. 11 is a block diagram of a system for facilitating the transmission of data associated with the treatment process of the at least one patient and a medical facility, in accordance with some embodiments.

FIG. 12 is a flowchart of a method to facilitate business architecture associated with the MyCareChip patient workflow review, in accordance with some embodiments.

FIG. 13 is a partial flowchart of a method to facilitate business architecture associated with MyCareChip patient workflow review, in accordance with some embodiments.

FIG. 14 is a partial flowchart of a method to facilitate business architecture associated with MyCareChip patient workflow review, in accordance with some embodiments.

FIG. 15 is a schematic of a technical architecture of a system associated with a hospital basic setup, in accordance with some embodiments.

FIG. 16 is a schematic of a system to facilitate transmission of data associated with the at least one patient, in accordance with some embodiments.

FIG. 17 is a block diagram of a computing device for implementing the methods disclosed herein, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems for facilitating the management of healthcare delivery processes, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, and at least one sensor. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview:

The present disclosure may describe systems for facilitating the management of healthcare delivery processes. Further, the system may facilitate access to healthcare and improving patient accountability. Further, the system may provide access to healthcare and increases patient involvement ultimately leading to more patient accountability from the patient's perspective. Further, the system may extend healthcare access and accountability beyond a medical facility through an interactive cloud-based patient database tool, application, and patient monitoring services. Further, the disclosed system may provide a vast array of services including, but not limited to, a healthcare system, billing services, and/or administrative and user-based Graphical User-Interface (GUI), etc.

Further, the system may include a cloud-based patient database and client capable of pulling registration information to store the patient's information required at any new facility, facilitating the globalized consolidation of a lengthy process. Further, the system may provide billing information pulled with reminders for the patient and quick tips for patients upon failing to pay as well as additional related financial information. Further, the system may store information on a wearable or implantable device to provide critical response, medical record number, and/or current medications while monitoring the patient's vitals. Further, the wearable or implantable device may be implanted in various locations on the body of the user, depending on the vitals which are being monitored. The wearable or implantable device may activate location services (e.g. GPS) through a mobile device to assist emergency responses or missing persons' cases. Further, the implantable device may be provided on a wearable device, such as a bracelet, or may be integrated on an external device, such as a credit card. Further, the system may provide administrative and user-based GUI to maintain and navigate information stored on a database associated with the system.

Nowadays, the medical care people receive in the healthcare system starts and ends at the local healthcare facility. While the traditional healthcare system has generally been efficient at providing necessary care to many people, the current healthcare system has many limitations. The current healthcare system often provides limited interaction between healthcare providers and people, resulting in occasional interactions when people get sick or require emergency care. Further, people are often left in the dark regarding the obligations and/or benefits associated with healthcare, such as medical bills, due to the lack of a clear channel of communication between the healthcare providers and people. Further, there are few or limited resources that allow for the continuous monitoring of people's health, leaving the responsibilities to people who oftentimes do not have the knowledge or experience to recognize health threats. In addition, people's medical history is often restricted and hard to access between different healthcare providers. If a person were to have a medical emergency, chances are the person can be taken to a healthcare facility where the medical history is not available. The medical history is crucial for healthcare as information such as previous medical conditions can provide valuable information to healthcare providers. Furthermore, people often fail to promptly receive healthcare in an efficient manner due to the lengthy process usually involved when requesting healthcare. These and many other limitations of the current healthcare system need and can be addressed.

An objective of the present invention is to provide systems and methods for facilitating access to healthcare and improving patient accountability. The present invention provides a cloud-based patient database that provides information on users from birth until death. With the assistance of a wearable or implantable device, the present invention stores critical user-identifying information, medications, allergies, disabilities, and/or precautions. The present invention further monitors vitals and activates emergency response based on complex algorithms that consider multiple variables such as current body velocity, pulse, blood pressure, temperature, and/or Oxygen levels to ultimately identify potential incidents and save users' lives.

Furthermore, the present invention provides healthcare assistance within a healthcare facility. Inside the healthcare facility, users can receive step by step directions to appointments or other health processes. The present invention will further be able to communicate with external healthcare systems. For example, the present invention can communicate with an existing healthcare system of a healthcare facility to alert when users have arrived at the healthcare facility and can provide users with all information, images, and reports regarding the users' care. The present invention will facilitate users' access to scheduling and rescheduling. The ability to schedule/reschedule can be done within the preset perimeters of the healthcare facility and the present invention can further provide alert-based tracking, thus ensuring that no time is ever wasted by the users due to a cancelation.

The present invention disclose may describe systems for facilitating access to healthcare and improving patient involvement to increase patient accountability during healthcare processes. The disclosed system, referred to as GOVIPCARE, may assist a patient in health processes and provides more access to healthcare and increasing a sense of involvement which may ultimately lead to more patient accountability, peace of mind, and the world's best critical health response. Further, the system may include a wearable or implantable device. Further, the wearable or implantable device, referred to as MyCareChip, may extend users' access, healthcare, and health monitoring beyond a healthcare facility.

Further, the MyCareChip may provide the ability to know family members are safe and secure, whether you may physically see them or not. Further, with the MyCareChip delicate real-world scenarios, such as lost child, may be solved. Further, the MyCareChip may facilitate identifying and monitoring disabled family members or senior citizens living alone. Further, solutions to delicate real-world scenarios may be made possible by providing instant notification and location to emergency response teams, location services actively managed through software application, secure link to full medical history, prior treatment, and imaging, safe-word and button activation for real-world emergencies, vital monitoring from your smartphone built to notify emergency response teams, when the user may need it most. Further, the disclosed system may include a way to hear the of the user voice in the event of a life-threatening emergency. Whether the threat to your life is something malicious or simply due to declining health mixed with disability, GOVIPCARE offers many helpful applications. Further, the disclosed system may include a way for an authorized authority to find the user, while providing the required documentation for an authorized physician to treat the user, faster than ever before. Further, the GOVIPCARE may accomplish to make the world safer and treatment faster for the user. Further, the MyCareChip may extend access, care, and monitoring beyond the doors of the care facility. This may be done by an interactive cloud-based patient database tool, web application, and patient monitoring service through an RFID implant or attachable device. Further, the GOVIPCARE may assist the user in the process from the patient's perspective while providing more access to care and a sense of involvement, ultimately leading to more patient accountability, peace of mind, and the world's best in critical response. Further, the GOVIP CARE application may offer a vast array of additional services such as access to patient history and secure cloud database, billing and scheduling modules, administrative and user-based mobile Graphical User Interface (GUI), GPS tracking through active mobile location services to provide world-class emergency response. Further, the GOVIP CARE may provide responsive CAD 3D mapping of participating hospitals with step by step directions to patient appointments. Further, the GOVIPCARE may include RFID microchip with vital monitoring technology with encrypted link to cloud storage unique to each patient further requiring the authorization and authentication through our physician verified and trusted users. Further, the system may facilitate the services through an interactive cloud-based patient database tool, software application, and health monitoring services provided by the wearable or implantable device. Further, the implantable device may include a wearable device which may be worn on parts of the body. Further, the implantable device may include a medical implant that may be positioned on plurality of location within the body. Further, the wearable or implantable device may store information for critical response which may include, but is not limited to, medical record number, current or prior medications, medical history, etc. Further, the wearable or implantable device may monitor the users' vitals. Further, the wearable or implantable device may provide location services. Further, the wearable or implantable device may activate the location services of the electronic device that may include the software application. Further, the wearable or implantable device may monitor biometrics associated with the user. Further, the wearable or implantable device may determine possible health threats to the user, upon monitoring the vitals associated with the user. Further, the wearable or implantable device may comprise voice-recognition capabilities. Further, the wearable or implantable device may be voice-activated for the users to utilize a safe word. Further, the safe word may include a combination of letters preset to trigger a predetermined process or action. Further, the wearable or implantable device may comprise integrated location services that may eliminate the need for the connection to an external electronic device and allowing accurate location directions. Further, the wearable or implantable device may facilitate the notification of family members of the user regarding the health and wellbeing of the users. Further, the wearable or implantable device may help the users locate a missing person or identify and monitor disabled family members. Further, the wearable or implantable device may facilitate the monitoring of the biometrics to improve the location services. Further, the signal may be generated using the wearable or implantable device upon detection of a health threat. Further, personalized and continuous monitoring of biometrics by the wearable or implantable device may facilitate the detection of health threats. Further, upon transferring of the user (patient) to a care facility where the user has never attended before, the wearable or implantable device may aid in the registration of the user in the healthcare system of the new care facility.

Further, the system may provide global registration and a plurality of databases. The system may meet Health Level Seven (HL7) standards to facilitate the pulling of registration information to store the users' information required to schedule at least one health process at any new healthcare facility, thus globalizing the consolidation of a lengthy process.

Further, the present disclosure, referred to as GOVIPCARE, may provide medical systems and methods for facilitating access to healthcare and improving patient accountability.

The disclosed system may record patient history, identity information, allergies, medications taken, disabilities, and so on associated with the patient. Further, the cloud, patient history, identity information, allergy's, medications taken, disabilities may be linked to database of prior imaging. Further, the disclosed system may monitor as many vitals as possible with a coded algorithm. Further, the disclosed system may be voice-activated to utilize a safe word when in trouble, whether it is physical or medical and using the phones and the software application's GPS may alert authorities. Further, the present disclosure may include an approach that may include the GPS on the chip. Further, the utilization of GPS may allow the CAD-based walkthrough in supported medical facilities as well. Further, the disclosed system may make the overall experience easier and more enjoyable.

Further, the talks about healthcare start and end at local hospital door. Further, every executive meeting is filled with the topics on how to cut down no waste and where to further streamline the process because we are dollars, not patients. Further, MYVIPCARE may extend care, access and accountability beyond the doors of the facility in an interactive cloud-based patient database tool, application, and patient monitoring service.

Further, the present disclosure may describe systems that may include cloud-based patient database and client. Further, the healthcare system HL7 interfacing may pull registration information to store the patient's information required to schedule at any new facility, globalized consolidation of a lengthy process. Further, the billing information may be pulled with reminders for the patient and quick tips when a patient doesn't pay. (denied care or worse). Further, the ability to store information on an implanted microchip for critical response, medical record number, current medications may monitor vitals associated with the patient. Further, algorithms written may activate GPS through mobile devices that may assist emergency response situations, medical or malicious, while also aiding in missing persons or lost child cases. Further, the administrative and user-based GUI may maintain maintenance and navigate information stored on the database. Further, the web-driven software application, IOS and Android application for phones with GPS assistance may facilitate billing alerts and tracking, notifications, and patient accountability. Further, the software application may facilitate scheduling that may allow patient to access the current healthcare system and re-schedule the appointments opening up further availability for patients who wish to be seen sooner. Further, the GPS tracking inside the care facility with transponders may provide step by step directions to the patients for appointment to arrive and notify staff, help desk, kiosk and freeing staff for more essential work. further, an emergency activation button may facilitate calling an automated code announcement (red, blue, white) with GPS location. Further, the patient vitals monitoring may alert local authorities and emergency response teams outside of the facility that may be linked to the software app and phone GPS. Further, the authorities may be alerted through microchip insert, watch or bracelet.

Further, the target market may not be bigger as healthcare and quality healthcare on a revolutionary scale may apply to everyone, everywhere. Further, competition in the cloud has imaging components and a few attempts may include attaching DICOM headers or patient criteria but none quite like ours. Further, the team may consist of everything that may be needed to build this kind of software application, an Information Technology certified professional in telecommunication, healthcare composite systems, interfacing and picture archiving and communication systems partnered with a professor of Radiology and founder of Torus Imaging Solutions, LLC.

Further, MyVIPCare is a software and web-based solution that may provide quality care and workflow improvements but taken from the patient's perspective. Further, the MyVIPCare may include a database, web-based mobile app, GPS transmitters and a patient monitoring component capable augmenting the patients care and overall experience at and beyond the medical facility that may be done by empowering the patients through easier access to care while holding the patients more accountable for the scheduling and billing by simplifying the overall process.

Further, the disclosed system may include a cloud-based patient database that may provide information on patients from birth until death much like Carfax does for your car. Further, the marketing aspect may revolutionize patient access to care and accountability. With the assistance of a microchip storing critical patient identifying information, medications, allergies, disabilities, and precautions to monitoring vitals and activating emergency response based on complex algorithms like current velocity, pulse, blood pressure, temperature and oxygen levels may ultimately identify and save lives.

To put this in perspective say your phone knows based on car mode your traveling 40 MPH and suddenly you stopped the chip notices the pulse is elevated and a decrease in oxygen in the blood is calculated. It then would send your vital information and location based on the GPS receiver in your phone to the local emergency response team based on the say 80% likelihood that you have been in a crash. (Alerting your phone first to engage or disengage the EMT response at any time). This is how MYVIPCARE may bring care beyond the doors of the hospital.

Further, the patient may receive GPS step by step directions to appointments inside the hospital. Further, the software application may let the current health care composite system know about the patient's arrival via the GPS transmitters and provide the patient with all information, images, and reports regarding care. The patient may have first-class access to scheduling and re-scheduling dates and times just as a technologist would see them. Cutting out the aggravated desk clerk as the patient may re-schedule or ask for a call back to be seen sooner. The ability to re-schedule or simply scan the date for earlier availability done within the preset perimeters of the scheduling hospital may be at the patients' disposal with alert based tracking ensuring that no time in facility is ever wasted due to a cancelation.

Further, the sales model may revolve around chips sold, installation and yearly service contracts as well as a yearly stipend for access based on year to date records of workload to what can be the largest cloud-based patient database in the world. Currently, it is planned for the cost of the app to download and use by the patient to be free.

Further, the GOVIP CARE may extend care, access, and accountability beyond the doors of the facility in an interactive cloud-based patient database tool, implantable or attachable storage device, and patient monitoring service that serves beyond the doors of any hospital.

Further, the disclosed system may include a mobile app for billing alerts and tracking, notifications, and patient accountability. Scheduling where a patient can access the current healthcare system for themselves and re-schedule their appointments opening further availability for patients who wish to be seen sooner. Further, GPS tracking inside the facility with transponders throughout providing mobile step by step directions to the patient's appointment with the capability to arrive and notify staff, help desk, kiosk and freeing staff for more essential work. An Emergency Activation button capable of calling an automated code announcement (red, blue, white) with GPS location. Further, the disclosed system may include wearable or Implantable RFID storage device. Further, patient vitals monitoring, capable of activating your devices location services to alert local authorities and emergency response teams anywhere, anytime phone services are available, using your phone GPS. This will be done through microchip insert, wearable device to include its own GPS in future testing for those who want the all in one package. Further, the GPS will be activated only if the written algorithms are breached, the panic button on the mobile device is pushed, or “safe word” spoken to your mobile device to voice activate emergency response.

Further, the enterprise data may include data that may be used by multiple departments or functions within the enterprise or is relevant to enterprise strategic planning, decision making or regulatory authorities. Further, the flow of data through the enterprise may be graphically represented in an information architecture diagram that may include elements such as an entity element, process element, data store element, and data flow element. Further, the entity element associated with the enterprise data flow may include source or destination of the data flow process. Further, the process element associated with the enterprise data flow may refer to manipulation and/or transformation of data. Further, the data storage element associated with the enterprise data flow may assist the process to store data for further usage. Further, the data flow element associated with the enterprise data flow may indicate the movement (or flow) of data.

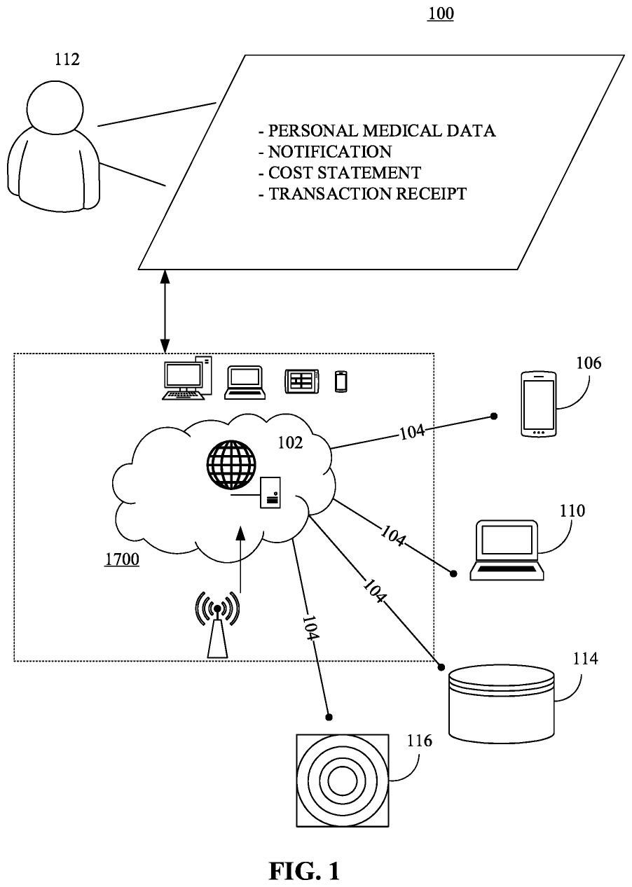

FIG. 1 is an illustration of an online platform 100 consistent with various embodiments of the present disclosure. By way of non-limiting example, the online platform 100 to facilitate the management of healthcare delivery processes may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 1700.

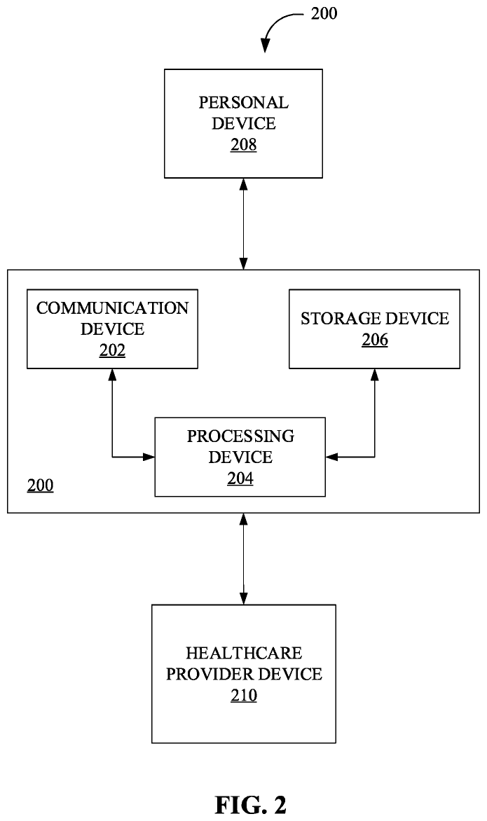

FIG. 2 is a block diagram of a system 200 for facilitating the management of healthcare delivery processes, in accordance with some embodiments. Accordingly, the system 200 may include a communication device 202, a processing device 204, a storage device 206.

Further, the communication device 202 may be configured for receiving at least one personal medical data from at least one personal device 208. Further, the at least one personal device 208 may be associated with a user. In an exemplary embodiment, the at least one personal device 208 may include, but is not limited to, an OLED display, a set of 10 k resistors, a micro USB, a 3.3+V regulator, a set of capacitors, a set of SMD tactile side switches, a 3.7V+ battery, a PCB board, a pulse sensor heart rate sensor (e.g. model number VUPN597), a battery charger chip (e.g. model number TP4056), a temperature sensor (e.g. model number TMP36), a Bluetooth LBE with a 2.4-GHZ transceiver, or any combination thereof. Further, the at least one personal device 208 configured for storing at least one personal medical data associated with the user. Further, the communication device 202 may be configured for transmitting at least one notification to at least one of a healthcare provider device 210 of at least one healthcare provider device 210 and a personal device 208 of the at least one personal device 208. Further, the at least one healthcare provider device 210 may be associated with at least one healthcare provider.

Further, the processing device 204 may be configured for analyzing the at least one personal medical data. Further, the processing device 204 may be configured for managing at least one healthcare delivery process based on the analyzing. Further, the managing may include generating the at least one notification.

Further, the storage device 206 may be configured for storing the at least one notification and the at least one personal medical data in a database.

Further, in some embodiments, the at least one personal device 208 may be configured to be worn in at least one body part of the user. Moreover, in these embodiments, the at least one personal device 208 may be configured as a computerized watch (i.e. “smart-watch) including, but not limited to, at least one micro-speaker, an antenna for a low-energy personal area network (e.g. Bluetooth), a management chip for the low-energy personal area network, a recharging port (e.g. a micro universal serial bus (USB) charger), and a rechargeable battery.

Further, in some embodiments, the at least one personal device 208 may be configured to be implanted in at least one body part of the user.

Further, in some embodiments, the at least one personal device 208 may include at least one biological sensor. Further, the at least one biological sensor may be configured to generate at least one biological sensor data associated with the user. Further, the at least one personal medical data may include at least one biological data.

Further, in some embodiments, the at least one personal device 208 may include at least one position sensor. Further, the at least one position sensor may be configured to generate at least one position data associated with the user. Further, the at least one personal medical data may include at least one position data.

Further, in some embodiments, the at least one personal device 208 may be configured to generate at least one user data. Further, the at least one user data may be associated with the user. Further, the communication device 202 may be further configured for receiving the at least one user data from the at least one personal device 208.

Further, in some embodiments, the storage device 206 may be further configured for retrieving at least one healthcare provider data associated with the at least one healthcare provider from the database based on the analyzing. Further, the at least one notification may include the at least one healthcare provider data.

Further, in some embodiments, the at least one personal device 208 may include at least one emergency sensor. Further, the at least one emergency sensor may be configured to generate at least one emergency sensor data associated at least one emergency situation. Further, the at least one personal medical data may include the at least one emergency sensor data. Further, the at least one notification may include an emergency alert.

Further, in some embodiments, the processing device 204 may be further configured for generating a confirmation query based on the analyzing. Further, the communication device 202 may be further configured for transmitting the confirmation query to the at least one personal device 208. Further, the communication device 202 may be configured for receiving a confirmation response from the at least one personal device 208. Further, the managing of the at least one healthcare delivery process may be based on the confirmation response.

Further, in some embodiments, the communication device 202 may be further configured for receiving a user indication associated with the user from the at least one healthcare provider device 210. Further, the at least one healthcare provider device 210 may include at least one identification sensor. Further, the at least one identification sensor may be configured to generate the user indication. Further, the storage device 206 may be further configured for retrieving at least one personal historical data associated with the user from the database. Further, the processing device 204 may be further configured for analyzing the at least one historical record. Further, the managing of the at least one healthcare delivery process may be based on the analyzing of the at least one personal historical data.

Further, in some embodiments, the managing may include allocating of at least one healthcare resources associated with the at least one healthcare provider for the user. Further, the at least one notification may include allocation data.

Further, in some embodiments, the managing may include navigating the user to at least one healthcare station of the at least one healthcare provider. Further, the user receives at least one healthcare service in the at least one healthcare station. Further, the at least one notification may include navigational data.

Further, in some embodiments, the managing may include navigating the at least one healthcare provider to the user. Further, the at least one notification may include user navigational data.

Further, in some embodiments, the managing may include registering the user with the at least one healthcare provider. Further, the at least one notification may include user profile data.

Further, in some embodiments, the storage device 206 may be further configured for retrieving at least one healthcare service data associated with the at least one healthcare provider from the database. Further, the processing device 204 may be further configured for analyzing the at least one healthcare service data and the personal medical data. Further, the managing may include scheduling at least one healthcare examination for the user with the at least one healthcare provider. Further, the at least one notification may include examination schedule data.

Further, in some embodiments, the storage device 206 may be further configured for retrieving at least one personal data associated with the user from the database. Further, the communication device 202 may be configured for receiving a cost statement associated with the user from the at least one healthcare provider device 210. Further, the communication device 202 may be configured for transmitting a transaction receipt to the at least one personal device 208. Further, the at least one personal device 208 may be configured to display the transaction receipt. Further, the processing device 204 may be configured for processing a transaction against the cost statement based on the at least one personal data. Further, the processing device 204 may be configured for generating the transaction receipt based on the processing.

Further, in some embodiments, the communication device 202 may be further configured for transmitting the cost statement to the at least one personal device 208.

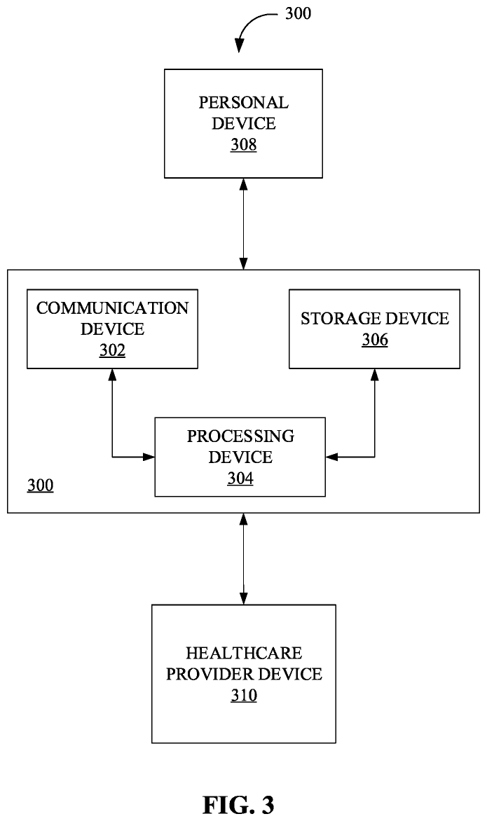

FIG. 3 is a block diagram of a system 300 for facilitating the management of healthcare delivery processes, in accordance with some embodiments. Accordingly, the system 300 may include a communication device 302, a processing device 304, a storage device 306.

Further, the communication device 302 may be configured for receiving at least one personal medical data from at least one personal device 308. Further, the at least one personal device 308 may be associated with a user. Further, the at least one personal device 308 configured for storing at least one personal medical data associated with the user. Further, the at least one personal device 308 may be configured to be implanted in at least one body part of the user. Further, the communication device 302 may be configured for transmitting at least one notification to at least one of a healthcare provider device 310 of at least one healthcare provider device 310 and a personal device 308 of the at least one personal device 308. Further, the at least one healthcare provider device 310 may be associated with at least one healthcare provider.

Further, the processing device 304 may be configured for analyzing the at least one personal medical data. Further, the processing device 304 may be configured for managing at least one healthcare delivery process based on the analyzing. Further, the managing may include generating the at least one notification.

Further, the storage device 306 may be configured for storing the at least one notification and the at least one personal medical data in a database.

Further, in some embodiments, the communication device 302 may be further configured for receiving a user indication associated with the user from the at least one healthcare provider device 310. Further, the at least one healthcare provider device 310 may include at least one identification sensor. Further, the at least one identification sensor may be configured to generate the user indication. Further, the storage device 306 may be further configured for retrieving at least one personal historical data associated with the user from the database. Further, the processing device 304 may be further configured for analyzing the at least one historical record. Further, the managing of the at least one healthcare delivery process may be based on the analyzing of the at least one personal historical data.

Further, in some embodiments, the storage device 306 may be further configured for retrieving at least one personal data associated with the user from the database. Further, the communication device 302 may be configured for receiving a cost statement associated with the user from the at least one healthcare provider device 310. Further, the communication device 302 may be configured for transmitting a transaction receipt to the at least one personal device 308. Further, the at least one personal device 308 may be configured to display the transaction receipt. Further, the processing device 304 may be configured for processing a transaction against the cost statement based on the at least one personal data. Further, the processing device 304 may be configured for generating the transaction receipt based on the processing.

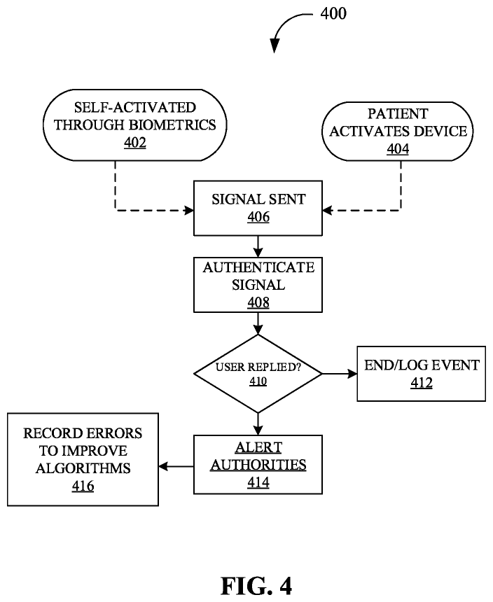

FIG. 4 is a flowchart of a method 400 to facilitate authentication of a signal associated with a wearable or implantable device, in accordance with some embodiments. Accordingly, at 402, the method 400 may include a step of generating, using the wearable or implantable device, the signal associated with a user based on monitoring of biometrics. Further, the user may include an individual that may want to use the software application. Further, the wearable or implantable device may include a wearable device which may be worn on parts of the body. Further, the implantable device may include a medical implant that may be positioned on plurality of location within the body. Further, the biometrics associated with the user may include blood pulse rate, heartbeat rate, blood pressure, etc. Further, the biometrics may be monitored by the wearable or implantable device. Further, upon detection of a health threat, the signal may be transmitted to a plurality of servers and databases.

Further, at 404, the method 400 may include a step of generating the signal using the wearable or implantable device. Further, the at least one patient may trigger the wearable or implantable device to generate the signal. Further, the implantable device may include a wearable device which may be worn on parts of the body. Further, the wearable or implantable device may include a medical implant that may be positioned on plurality of location within the body. Further, the wearable or implantable device may store information for critical response which may include, but is not limited to, medical record number, current or prior medications, medical history, etc. Further, the wearable or implantable device may monitor the users' vitals. Further, the wearable or implantable device may provide location services (e.g. geospatial positioning system (GPS) capabilities). Further, the wearable or implantable device may activate the location services of the electronic device that may include the software application. Further, the wearable or implantable device may monitor biometrics associated with the user. Further, the wearable or implantable device may determine possible health threats to the user, upon monitoring the vitals associated with the user. Further, the wearable or implantable device may comprise voice-recognition capabilities. Further, the wearable or implantable device may be voice-activated for the users to utilize a safe word (e.g. “siren, rape”, “siren, how is dad?”, or the name of a family member). Further, the safe word may include a combination of letters preset to trigger a predetermined process or action. Further, the wearable or implantable device may comprise integrated location services that may eliminate the need for the connection to an external electronic device and allowing accurate location directions. Further, the wearable or implantable device may facilitate the notification of family members of the user regarding the health and wellbeing of the users. Further, the wearable or implantable device may help the users locate a missing person or identify and monitor disabled family members. Further, the wearable or implantable device may facilitate the monitoring of the biometrics to improve the location services, wherein the biometrics may include, but is not limited to, heart rate and/or bodily temperature. Further, the signal may be generated using the wearable or implantable device upon detection of a health threat. Further, personalized and continuous monitoring of biometrics by the wearable or implantable device may facilitate the detection of health threats. Further, upon transferring of the user (i.e. patient) to a care facility where the user has never attended before, the wearable or implantable device may aid in the registration of the user in the healthcare system of the new care facility.

Further, at 406, the method 400 may include a step of transmitting the signal to a plurality of servers and plurality of databases. Further, a database of the plurality of database may facilitate receiving and storing signal information associated with the signal generated by the patient. Further, the signal information may include details of the at least one patient, health threat details, etc. Further, the details of the at least one patient may include name, age, address, medical history, etc. Further, the medical history may include a medical disorder, duration of medical disorder, and so on. Further, the health threat details may include name of the health threat.

Further, at 408, the method 400 may include a step of authenticating the signal associated with the at least one patient.

Further, at 410, the method 400 may include a step of receiving a response from the at least one patient to facilitate the authentication of the signal. Further, the signal may be authenticated by the at least one patient, upon utilizing a safe word. Further, the authentication of the signal may be performed manually upon contacting at least one patient and receiving a confirmation associated with wellbeing of the at least one patient. Further, the signal may be authenticated upon recognizing the voice provided by the wearable or implantable device.

Further, at 412, the method 400 may include a step of terminating and logging the event associated with the health risk of the at least one patient.

Further, at 414, the method 400 may include a step of transmitting an alert to authorities. Further, the alert may be associated with the health threat. Further, the authorities may include a hospital, a police department, a fire department, etc.

Further, at 416, the method 400 may include a step of recording errors to improve algorithms. Further, the algorithms may include a set of instructions.

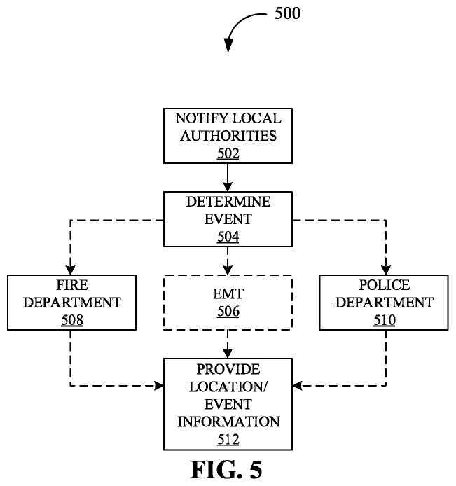

FIG. 5 is a flowchart of a method 500 to facilitate communication between the at least one patient and the authority, in accordance with some embodiments. Accordingly, at 502, the method 500 may include a step of notifying the authority based on the health threat.

Further, at 504, the method 500 may include a step of determination of the health threat (or event).

Further, at 506, the method 500 may include a step of notifying the Emergency Medical Technician (EMT) of the health threat.

Further, at 508, the method 500 may include a step of notifying the fire department of the health threat.

Further, at 510, the method 500 may include a step of notifying the police department of the health threat.

Further, at 512, the method 500 may include a step of transmitting details associated with the health threat. Further, the details of the at least one patient may include name, age, address, etc. Further, the health threat details may include type of the health threat, severity of the health threat, etc. Further, upon visiting the at least one patient, the authority may link with the wearable or implantable device to identify the at least one patient and retrieve the details of the at least one patient.

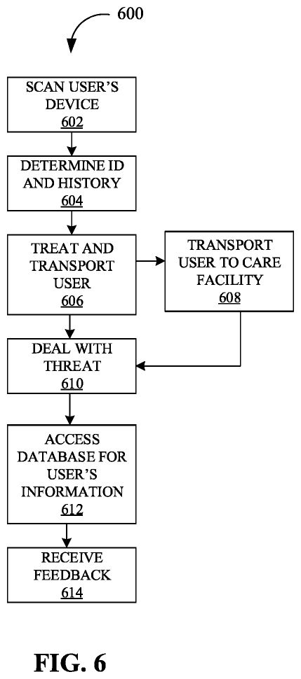

FIG. 6 is a flowchart of a method 600 to facilitate communication between the at least one patient and the authority to obtain the details of the at least one patient to provide treatment, in accordance with some embodiments. Accordingly, at 602, the method 600 may include a step of scanning patient device associated with the at least one patient. Further, the patient device may include a mobile, a tablet, a smartphone, a laptop, a personal computer, and so on.

Further, at 604, the method 600 may include a step of determining ID (Identification) number associated with the at least one patient. Further, history of the at least one patient may be retrieved using the ID number. Further, the history may include details of the at least one patient.

Further, at 606, the method 600 may include a step of providing treatment and transporting the at least one patient.

Further, at 608, the method 600 may include a step of transporting the at least one patient to a care facility. Further, the care facility may include a hospital.

Further, at 610, the method 600 may include a step of dealing with the health threat and treating the health threat.

Further, at 612, the method 600 may include a step of accessing a database to retrieve details of the at least one patient.

Further, at 614, the method 600 may include a step of receiving a feedback from the at least one patient.

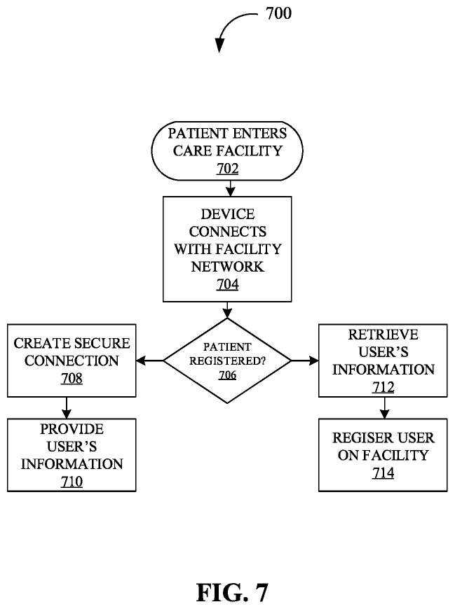

FIG. 7 is a flowchart of a method 700 to facilitate registration of the at least one patient, in accordance with some embodiments. Accordingly, at 702, the method 700 may include a step of arrival of the at least one patient to the care facility.

Further, at 704, the method 700 may include a step of connecting the wearable or implantable device with the facility network.

Further, at 706, the method 700 may include a step of verifying the registration of the at least one patient. Further, the at least one patient may be automatically detected upon verification of the registration of the at least one patient.

Further, at 708, the method 700 may include a step of establishing a secure connection with the facility network.

Further, at 710, the method 700 may include a step of receiving the details of the at least one patient and providing to the care facility (or authority).

Further, at 712, the method 700 may include a step of retrieving the details of the at least one patient from the at least one patient for registration on the facility.

Further, at 714, the method 700 may include a step of registration of the at least one patient. Further, upon registration of the at least one patient, a secure connection may be established with the facility network and provide the details of the at least one patient to the care facility. Further, the care facility may link with the wearable or implantable device on the at least one patient to access necessary information during the healthcare facility stay.

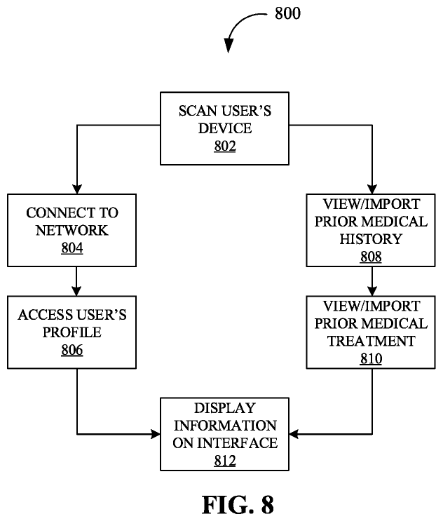

FIG. 8 is a flowchart of a method 800 to facilitate the retrieving of the details of the at least one patient from the wearable or implantable device, in accordance with some embodiments. Accordingly, at 802, the method 800 may include a step of scanning the wearable or implantable device to retrieve the details of the at least one patient.

Further, at 804, the method 800 may include a step of establishment of secure connections for the transfer of data that may meet Health Level Seven (HL7) standards.

Further, at 806, the method 800 may include a step of accessing the profile associated with the user.

Further, at 808, the method 800 may include a step viewing prior medical history associated with the user.

Further, at 810, the method 800 may include a step of viewing prior medical treatment associated with the user.

Further, at 812, the method 800 may include a step of displaying information on the interface for interaction of the user.

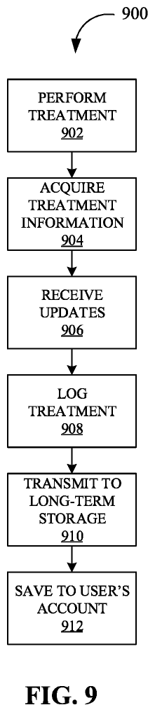

FIG. 9 is a flowchart of a method 900 to facilitate compiling and recording of treatment processes associated with health care of the at least one patient, in accordance with some embodiments. Accordingly, at 902, the method 900 may include a step of performing treatment processes of the at least one patient. Further, the treatment process may include imaging, testing result, examination study, etc. associated with the at least one patient.

Further, at 904, the method 900 may include a step of obtaining treatment information associated with the at least one patient. Further, the treatment information may include the information associated with the treatment processes.

Further, at 906, the method 900 may include a step of receiving updates associated with the treatment information of the at least one patient.

Further, at 908, the method 900 may include a step of compiling a log and/or report associated with the treatment information.

Further, at 910, the method 900 may include a step of transmitting the report to long term storage (such as database). Further, the treatment information associated with the at least one patient may be accessed from the software application associated with the disclosed system.

Further, at 912, the method 900 may include a step of transmitting the report to the account associated with the at least one patient.

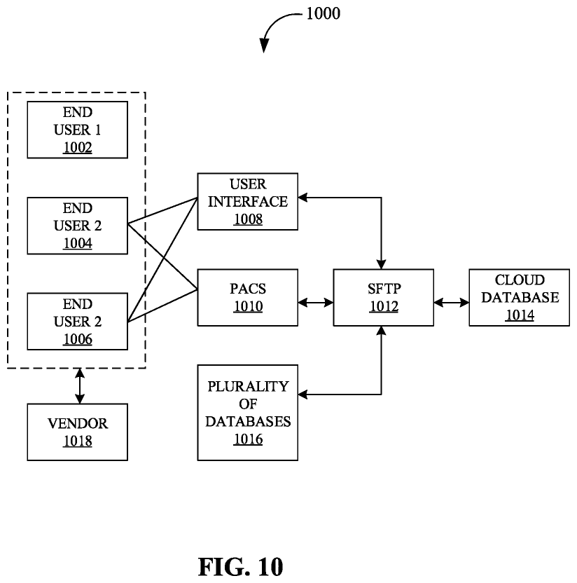

FIG. 10 is a block diagram of a system 1000 for facilitating the exchange of data over a plurality of data transfer protocols and standards, in accordance with some embodiments. Accordingly, the system 1000 may include a first end-user 1002, a second end-user 1004, third end-user 1006 that may want to transmit data. Further, the system 1000 may include a user interface 1008 associated with the software application that may transmit data over the plurality of data transfer protocols and standards. Further, the system 1000 may include Picture Archiving and Communication System (PACS) 1010 that may facilitate the transmission of data. Further, the system 1000 may include Secure Socket Shell File Transfer Protocol (SFTP) 1012 that may facilitate the transmission of data. Further, the system 1000 may include a cloud database 1014 that may store the data associated with the software application. Further, the software application may provide administrative and end-user Graphic User Interface (GUI) to maintain and navigate the details of the at least one patient stored on the database. Further, the system 1000 may include a plurality of databases 1016 that may facilitate storage of data. Further, at 1018, the system 1000 may include a vendor that may provide support.

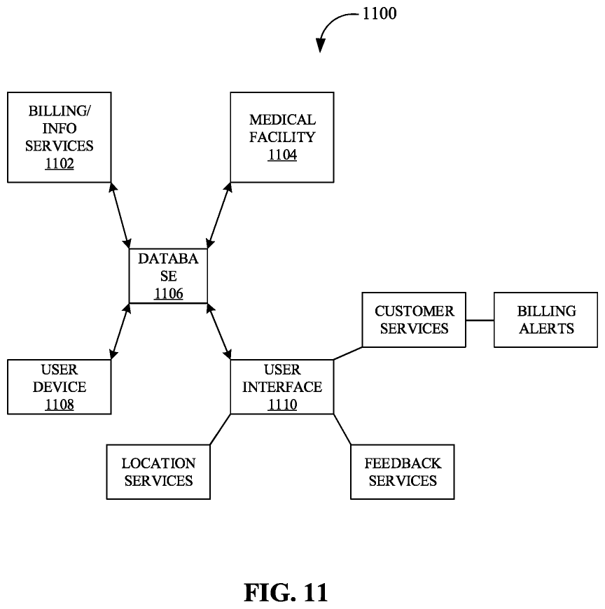

FIG. 11 is a block diagram of a system 1100 for facilitating the transmission of data associated with the treatment process of the at least one patient and a medical facility, in accordance with some embodiments. Accordingly, the system 1100 may include billing information/services 1102 associated with the at least one patient. Further, the billing services 1102 may provide tips to the at least one patient upon failing to pay money for the bills associated with the treatment processes.

Further, the system 1100 may include a medical facility 1104 (such as hospital) that may perform the treatment processes associated with the at least one patient.

Further, the system 1100 may include a database 1106. Further, the database 1106 may facilitate the storage of the treatment information associated with the at least one patient and the medical facility.

Further, the system 1100 may include a user device 1108 associated with the user. Further, the user device may include a smartphone, a mobile, a tablet, a laptop, a personal computer, and so on.

Further, the system 1100 may include a user interface 1110 associated with the software application. Further, the software application may assist the at least one patient to track the bills and provide notification and user accountability. Further, the software application can be provided to be utilized in different electronic devices. The software application can be provided as a mobile application, desktop application, cloud application, etc. Further, the software application may be accessed through a third-party application, such as web browser or search engine, in the form of a widget, extension, add-on, etc. Further, the software application may facilitate scheduling, so the users may access the current healthcare system and reschedule appointments with little or no assistance, thus opening further availability for users who wish to be seen sooner. Further, the software application may provide location tracking inside the care facility. A plurality of transponders provided throughout the healthcare facility in communication with the software application may provide step by step directions to the users' software application before and after the appointment. Further, the appointment may include a request to meet doctor in the care facility. Further, the software application may notify the users, upon arrival of at least one user of the user at the care facility that may allow staff to perform more essential work. Furthermore, the software application may activate the authority and emergency response teams outside of the care facility. Further, the software application may facilitate access to healthcare and improve user accountability. Further, the user interface 1110 may facilitate providing customer services, feedback services, location services, etc. Further, the customer services may include providing billing alerts associated with the bills associated with the treatment processes.

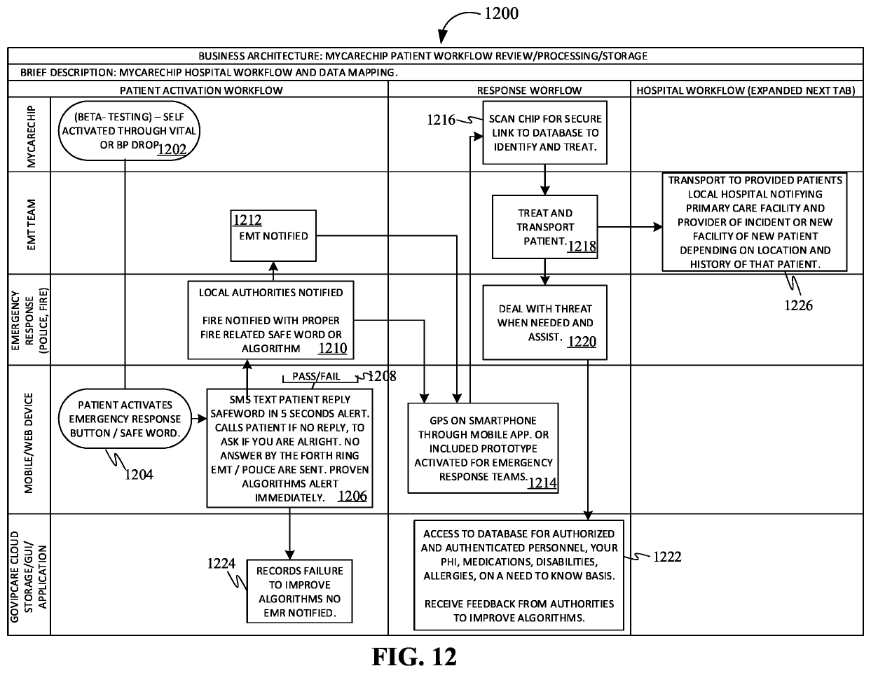

FIG. 12 is a flowchart of a method 1200 to facilitate business architecture associated with the MyCareChip patient workflow review, in accordance with some embodiments. Accordingly, at 1202, the method 1200 may include beta-testing that may be self-activated based on vital or Blood Pressure (BP) drop using the MyCareChip.

Further, at 1204, the method 1200 may include a step of activation, using the user device, of emergency response button/safe word.

Further, at 1206, the method 1200 may include a step of replying, using the user device, safeword through SMS by the at least one patient. Further, the at least one patient may reply in 5 seconds. Further, upon failing to send the reply, the at user may receive a call to confirm the health of the at least one patient. Further, Emergency Medical Technician and/or police may be sent to the at least one patient upon no answer by the fourth ring. Further, algorithms may facilitate alerting of the police/EMT.

Further, at 1208, the method 1200 may include a confirmation associated with the replying by the at least one patient. Further, the confirmation may include pass or fail.

Further, at 1210, the method 1200 may include a step of notifying the emergency response local authority (or authority). Further, the emergency response local authority may include the fire department, police department, and so on. Further, the fire department may be notified with safe word or algorithm associated with fire department.

Further, at 1212, the method 1200 may include a step of notifying the Emergency Medical Technician.

Further, at 1214, the method 1200 may include a step of activation of the Global Positioning System (GPS) using the at least one patient device. Further, GPS on the user device may be activated through the software application or prototype for emergency response authority/team.

Further, at 1216, the method 1200 may include a step of scanning the MyCareChip for secure linking to the database to identify and treat.

Further, at 1218, the method 1200 may include a step of treating and transporting the at least one patient by a team associated with the EMT.

Further, at 1220, the method 1200 may include a step of dealing and providing assistance with health threat upon requirement.

Further, at 1222, the method 1200 may include a step of providing access to database for authorized and authenticated personnel for Protected Health Information (PHI), medications, disabilities, allergies associated with the at least one patient for a need to know basis. Further, the feedback may be received from the emergency response local authority to improve algorithms.

Further, after 1206, at 1224, the method 1200 may include recording failure to improve algorithms. Further, the failure may be associated with the at least one patient that may not be able to send the reply. Further, no EMR may be notified.

Further, after 1218, at 1226, the method 1200 may include a step of notifying the primary care facility and/or provider of incident or new facility of a new patient based on location and history of the new patient.

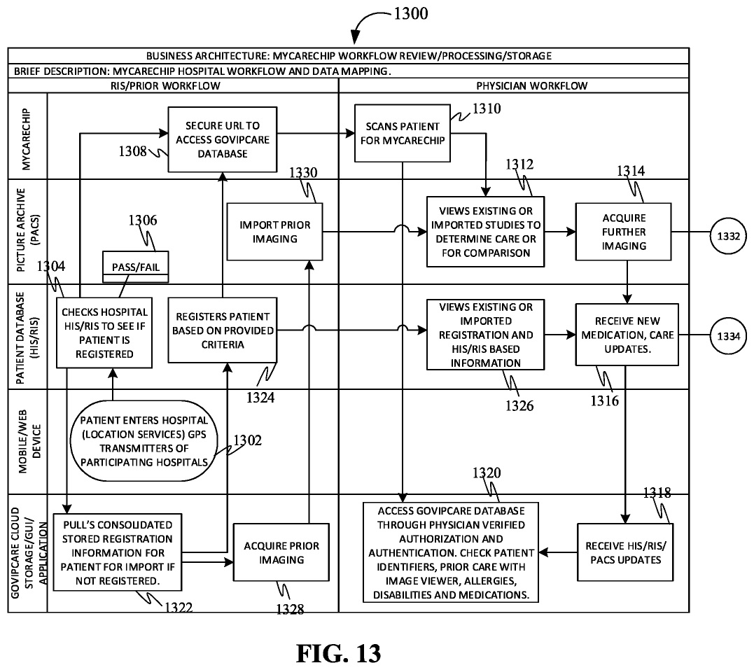

FIG. 13 is a partial flowchart of a method 1300 to facilitate business architecture associated with MyCareChip workflow review, in accordance with some embodiments.

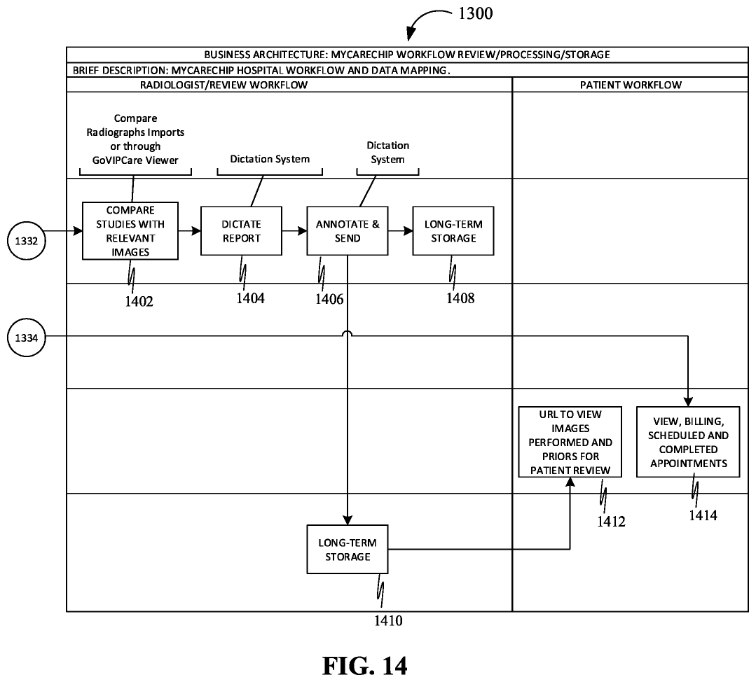

FIG. 14 is a partial flowchart of a method 1300 to facilitate business architecture associated with MyCareChip workflow review, in accordance with some embodiments.

Accordingly, at 1302, the method 1300 may include a step of determining, using the at least one patient device, the arrival of the at least one patient into the hospital (or emergency local authority). Further, the determination of arrival of the at least one patient may be facilitated by the GPS transmitters of the hospital.

Further, at 1304, the method 1300 may include a step of verifying, using a patient database (such as Hospital Information System (HIS), Radiology Information System (RIS), etc.) associated with the hospital, the registration of the at least one patient.

Further, at 1306, the method 1300 may include confirmation associated with the replying by the at least one patient. Further, the confirmation may include pass or fail.

Further, at 1308, the method 1300 may include a step of establishing, using the MyCareChip, secure Uniform Resource Locator (URL) to access the database.

Further, at 1310, the method 1300 may include a step of scanning the at least one patient for the MyCareChip.

Further, at 1312, the method 1300 may include a step of displaying, using the Picture Archive and Communication System (PACS), existing or imported studies to determine care of the at least one patient, or for comparison. Further, the existing or imported studies may be associated with the at least one patient.

Further, at 1314, the method 1300 may include a step of retrieving, using the PACS, further imaging associated with the at least one patient.

Further, after 1314, at 1332, the method 1300 may include a step of continuation to 1402.

Further, at 1402, the method 1300 may include a step of comparing, using the PACS, radiograph imports or through GoVIPCare Viewer.

Further, at 1404, the method 1300 may include a step of dictating a report associated with the at least one patient.

Further, at 1406, the method 1300 may include a step of annotation and sending the report.

Further, at 1408, the method 1300 may include a step of storing the report for long term.

Further, after 1406, at 1410, the method 1300 may include a step of storing the report for the long term.

Further, at 1412, the method 1300 may include a step of generating a URL to view images performed and prior images associated with the at least one patient for reviewing the at least one patient.

Further, after 1314, at 1316, the method 1300 may include a step of receiving, using the patient database, new medication, care updates associated with the at least one patient.

Further, after 1316, at 1334, the method 1300 may include a step of continuation to 1414.

Further, at 1414, the method 1300 may include a step of displaying the bills associated with the treatment processes, scheduled appointments, and completed appointments using the at least one user device.

Further, after 1316, at 1318, the method 1300 may include a step of receiving, using the GOVIPCARE cloud storage, updates associated with the HIS, RIS, PACS for the at least one patient.

Further, at 1320, the method 1300 may include a step of accessing, using the GOVIPCARE cloud storage, to check patient identifiers, prior care with image viewer, allergies, disabilities, and medications, upon physician verified authorization and authentication.

Further, after 1304, at 1322, the method 1300 may include a step of retrieving, using the GOVIPCARE cloud storage, consolidated stored registration information for importing the at least one patient that may not be registered. Further, the consolidated stored registration information may be associated with the at least one patient.

Further, at 1324, the method 1300 may include a step of registering, using the patient database, the at least one patient based on provided criteria (such as the consolidated stored registration information). Further, the step 1324 may be followed by step 1308.

Further, after 1324, at 1326, the method 1300 may include a step of displaying, using the patient database, existing or imported registration of the at least one patient. Further, the HIS and RIS based information associated with the at least one patient may be displayed. Further, the step 1326 may be followed by step 1316.

Further, after 1322, at 1328, the method 1300 may include retrieving, using the GOVIPCARE cloud storage, prior imaging associated with the at least one patient.

Further, at 1330, the method 1300 may include importing, using the PACS, prior imaging associated with the at least one patient. Further, the step 1330 may be followed by 1312.

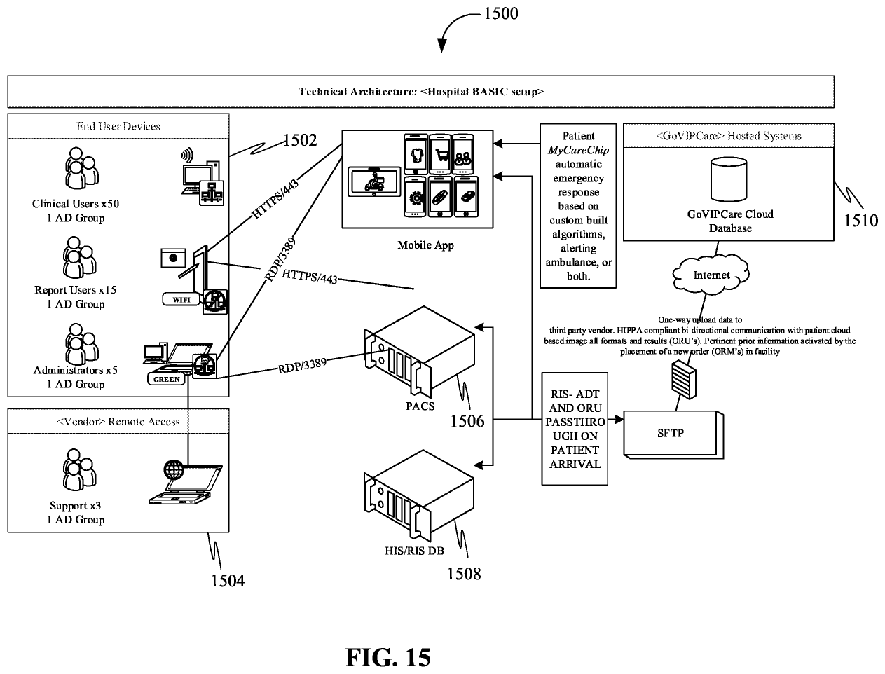

FIG. 15 is a schematic of a technical architecture of a system 1500 associated with a hospital basic setup, in accordance with some embodiments. Accordingly, the system 1500 includes end-user devices 1502, remote access for vendors 1504, Picture Archive and Communication System (PACS) 1506, Hospital Information System (HIS)/Radiological Information System (RIS) Database 1508, GOVIPCARE cloud database 1510. The end-user devices 1502 may include a smartphone, a tablet, a personal computer, a laptop, and so on. Further, the end-user devices 1502 may include an application for end-user interaction. Further, the MyCareChip associated with the at least one patient may transmit automatic emergency response based on custom-built algorithms that may alert ambulance, police or both. Further, the Radiological Information System 1508—Admissions Discharges Transfers (ADT) and Observation Results (ORU) may pass through on patient arrival. Further, data may be uploaded to a third-party vendor. Further, Health Insurance Portability and Accountability Act (HIPPA) compliant bi-directional communication with patient cloud-based image of all formats and results (ORU's). Further, pertinent prior information may be activated by the placement of a new Order entry (ORM's) in the care facility.

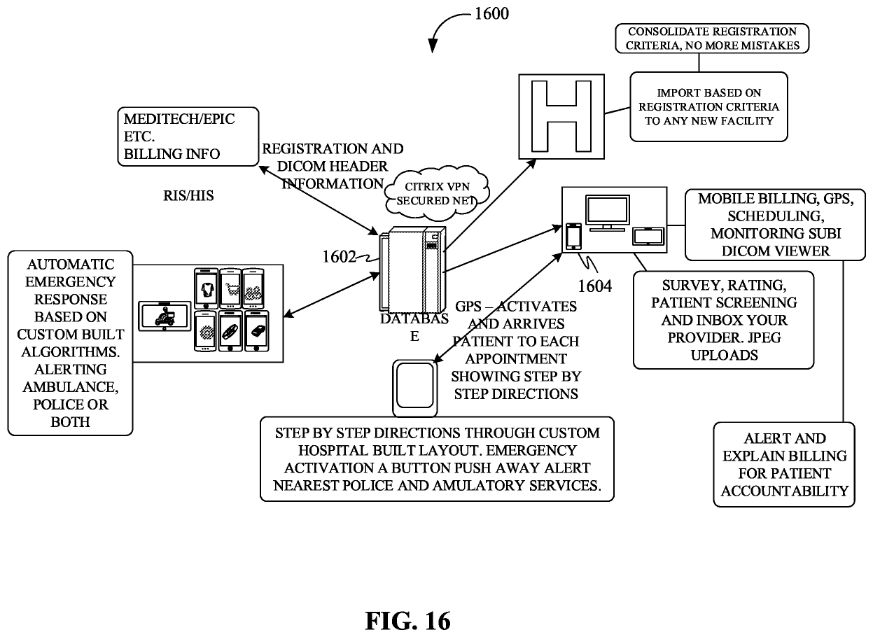

FIG. 16 is a schematic of a system 1600 to facilitate transmission of data associated with the at least one patient, in accordance with some embodiments. Accordingly, the system 1600 may include a database 1602 and a user device 1604. Further, the user device 1604 may include a smartphone, a tablet, a personal computer, a laptop, and so on. Further, the user device may be associated with a user that may want to store data. Further, the database 1602 may facilitate storage of data over a Virtual Private Network (VPN) such as Citrix VPN Secured Net. Further, the user device may be communicatively coupled with the database. Further, the Meditech/Epic, etc. and billing information associated with the RIS/HIS 1508 may be transmitted to the database. Further, registration and DICOM (Digital Imaging and Communication in Medicine) may be transmitted to the database. Further, the emergency button may alert nearest police and amulatory services. Further, the data associated with the GPS to provide step by step instructions through custom hospital built layout may be transmitted to the user device. Further, the automatic emergency response based on custom built algorithms to alert police, ambulance or both may be transmitted to the database. Further, the survey, ratings, patient screening, and JPEG uploads may be transmitted to the user device. Further, the data associated with the mobile billing, GPS, scheduling, monitoring SUBI DICOM viewer. Further, the alert and billing for patient accountability may be transmitted to the user device. Further, the consolidated registration criteria may facilitate import based on registration criteria to a new facility (such as hospital) without mistakes.

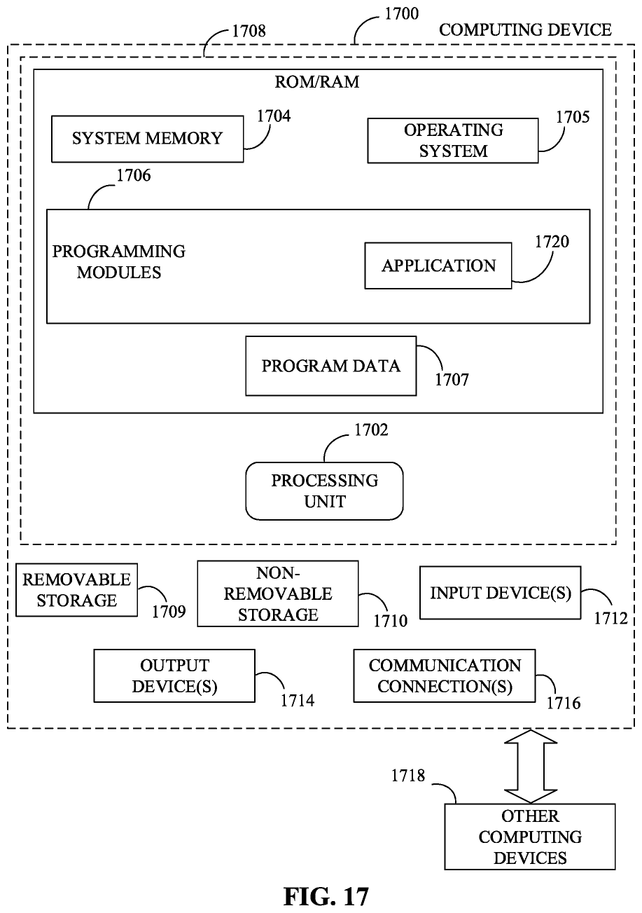

With reference to FIG. 17, a system consistent with an embodiment of the disclosure may include a computing device or cloud service, such as computing device 1700. In a basic configuration, computing device 1700 may include at least one processing unit 1702 and a system memory 1704. Depending on the configuration and type of computing device, system memory 1704 may comprise, but is not limited to, volatile (e.g. random-access memory (RAM)), non-volatile (e.g. read-only memory (ROM)), flash memory, or any combination. System memory 1704 may include operating system 1705, one or more programming modules 1706, and may include a program data 1707. Operating system 1705, for example, may be suitable for controlling computing device 1700's operation. In one embodiment, programming modules 1706 may include image-processing module, machine learning module. Furthermore, embodiments of the disclosure may be practiced in conjunction with a graphics library, other operating systems, or any other application program and is not limited to any particular application or system. This basic configuration is illustrated in FIG. 17 by those components within a dashed line 1708.

Computing device 1700 may have additional features or functionality. For example, computing device 1700 may also include additional data storage devices (removable and/or non-removable) such as, for example, magnetic disks, optical disks, or tape. Such additional storage is illustrated in FIG. 17 by a removable storage 1709 and a non-removable storage 1710. Computer storage media may include volatile and non-volatile, removable and non-removable media implemented in any method or technology for storage of information, such as computer-readable instructions, data structures, program modules, or other data. System memory 1704, removable storage 1709, and non-removable storage 1710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1700. Any such computer storage media may be part of device 1700. Computing device 1700 may also have input device(s) 1712 such as a keyboard, a mouse, a pen, a sound input device, a touch input device, a location sensor, a camera, a biometric sensor, etc. Output device(s) 1714 such as a display, speakers, a printer, etc. may also be included. The aforementioned devices are examples and others may be used.

Computing device 1700 may also contain a communication connection 1716 that may allow device 1700 to communicate with other computing devices 1718, such as over a network in a distributed computing environment, for example, an intranet or the Internet.

Communication connection 1716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1704, including operating system 1705. While executing on processing unit 1702, programming modules 1706 (e.g., application 1720 such as a media player) may perform processes including, for example, one or more stages of methods, algorithms, systems, applications, servers, databases as described above. The aforementioned process is an example, and processing unit 1702 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A system for facilitating the management of healthcare delivery processes, the system comprising:
a communication device configured for:
receiving at least one personal medical data from at least one personal device, wherein the at least one personal device is associated with a user, wherein the at least one personal device configured for storing at least one personal medical data associated with the user;
transmitting at least one notification to at least one of a healthcare provider device of at least one healthcare provider device and a personal device of the at least one personal device, wherein the at least one healthcare provider device is associated with at least one healthcare provider;
a processing device configured for:
analyzing the at least one personal medical data;
managing at least one healthcare delivery process based on the analyzing, wherein the managing comprises generating the at least one notification; and
a storage device configured for storing the at least one notification and the at least one personal medical data in a database.

2. The system of claim 1, wherein the at least one personal device is configured to be worn in at least one body part of the user.

3. The system of claim 1, wherein the at least one personal device is configured to be implanted in at least one body part of the user.

4. The system of claim 1, wherein the at least one personal device comprises at least one biological sensor, wherein the at least one biological sensor is configured to generate at least one biological sensor data associated with the user, wherein the at least one personal medical data comprises at least one biological data.

5. The system of claim 1, wherein the at least one personal device comprises at least one position sensor, wherein the at least one position sensor is configured to generate at least one position data associated with the user, wherein the at least one personal medical data comprises at least one position data.

6. The system of claim 1, wherein the at least one personal device is configured to generate at least one user data, wherein the at least one user data is associated with the user, wherein the communication device is further configured for receiving the at least one user data from the at least one personal device.

7. The system of claim 1, wherein the storage device is further configured for retrieving at least one healthcare provider data associated with the at least one healthcare provider from the database based on the analyzing, wherein the at least one notification comprises the at least one healthcare provider data.

8. The system of claim 1, wherein the at least one personal device comprises at least one emergency sensor, wherein the at least one emergency sensor is configured to generate at least one emergency sensor data associated at least one emergency situation, wherein the at least one personal medical data comprises the at least one emergency sensor data, wherein the at least one notification comprises an emergency alert.

9. The system of claim 1, wherein the processing device is further configured for generating a confirmation query based on the analyzing, wherein the communication device is further configured for:
transmitting the confirmation query to the at least one personal device; and
receiving a confirmation response from the at least one personal device, wherein the managing of the at least one healthcare delivery process is based on the confirmation response.

10. The system of claim 1, wherein the communication device is further configured for receiving a user indication associated with the user from the at least one healthcare provider device, wherein the at least one healthcare provider device comprises at least one identification sensor, wherein the at least one identification sensor is configured to generate the user indication, wherein the storage device is further configured for retrieving at least one personal historical data associated with the user from the database, wherein the processing device is further configured for analyzing the at least one historical record, wherein the managing of the at least one healthcare delivery process is based on the analyzing of the at least one personal historical data.

11. The system of claim 1, wherein the managing comprises allocating of at least one healthcare resources associated with the at least one healthcare provider for the user, wherein the at least one notification comprises allocation data.

12. The system of claim 1, wherein the managing comprises navigating the user to at least one healthcare station of the at least one healthcare provider, wherein the user receives at least one healthcare service in the at least one healthcare station, wherein the at least one notification comprises navigational data.

13. The system of claim 1, wherein the managing comprises navigating the at least one healthcare provider to the user, wherein the at least one notification comprises user navigational data.

14. The system of claim 1, wherein the managing comprises registering the user with the at least one healthcare provider, wherein the at least one notification comprises user profile data.

15. The system of claim 1, wherein the storage device is further configured for retrieving at least one healthcare service data associated with the at least one healthcare provider from the database, wherein the processing device is further configured for analyzing the at least one healthcare service data and the personal medical data, wherein the managing comprises scheduling at least one healthcare examination for the user with the at least one healthcare provider, wherein the at least one notification comprises examination schedule data.

16. The system of claim 1, wherein the storage device is further configured for retrieving at least one personal data associated with the user from the database, wherein the communication device is configured for:
receiving a cost statement associated with the user from the at least one healthcare provider device;
transmitting a transaction receipt to the at least one personal device, wherein the at least one personal device is configured to display the transaction receipt, wherein the processing device is configured for:
processing a transaction against the cost statement based on the at least one personal data; and
generating the transaction receipt based on the processing.

17. The system of claim 16, wherein the communication device is further configured for transmitting the cost statement to the at least one personal device.

18. A system for facilitating the management of healthcare delivery processes, the system comprising:
a communication device configured for:
receiving at least one personal medical data from at least one personal device, wherein the at least one personal device is associated with a user, wherein the at least one personal device configured for storing at least one personal medical data associated with the user, wherein the at least one personal device is configured to be implanted in at least one body part of the user;
transmitting at least one notification to at least one of a healthcare provider device of at least one healthcare provider device and a personal device of the at least one personal device, wherein the at least one healthcare provider device is associated with at least one healthcare provider;
a processing device configured for:
analyzing the at least one personal medical data;
managing at least one healthcare delivery process based on the analyzing, wherein the managing comprises generating the at least one notification; and
a storage device configured for storing the at least one notification and the at least one personal medical data in a database.

19. The system of claim 18, wherein the communication device is further configured for receiving a user indication associated with the user from the at least one healthcare provider device, wherein the at least one healthcare provider device comprises at least one identification sensor, wherein the at least one identification sensor is configured to generate the user indication, wherein the storage device is further configured for retrieving at least one personal historical data associated with the user from the database, wherein the processing device is further configured for analyzing the at least one historical record, wherein the managing of the at least one healthcare delivery process is based on the analyzing of the at least one personal historical data.

20. The system of claim 18, wherein the storage device is further configured for retrieving at least one personal data associated with the user from the database, wherein the communication device is configured for:
receiving a cost statement associated with the user from the at least one healthcare provider device;
transmitting a transaction receipt to the at least one personal device, wherein the at least one personal device is configured to display the transaction receipt, wherein the processing device is configured for:
processing a transaction against the cost statement based on the at least one personal data; and
generating the transaction receipt based on the processing.
